(19) United States
(12) Patent Application Publication
(10) Pub. No.: US 2026/0224195 A1
(43) Pub. Date: Aug. 6, 2026

(54) CALCIUM REVERB INDICATION FOR INTRAVASCULAR IMAGING GRAPHICAL USER INTERFACE

(71) Applicant: Boston Scientific Scimed, Inc., Maple Grove, MN (US)

(72) Inventors: Viola Kim Holman Narveson, Crystal, MN (US); Haruka Imura, Maple Grove, MN (US); Greta Evangeline O'Brien, Austin, TX (US); Valerie Alissa Engh, Inver Grove Heights, MN (US)

(73) Assignee: Boston Scientific Scimed, Inc., Maple Grove, MN (US)

(21) Appl. No.: 19/530,066

(22) Filed: Feb. 4, 2026

Related U.S. Application Data
(60) Provisional application No. 63/753,858, filed on Feb. 4, 2025.

(51) Int. Cl. A61B 8/12; A61B 8/00

(52) U.S. Cl. CPC A61B 8/12; A61B 8/461; A61B 8/5223

(57) ABSTRACT

The present disclosure provides a graphical user interface (GUI) arranged to convey information related to the IVUS images and calcium reverb detected in a vessel represented in the IVUS images. The GUIs can be generated to include a cross-section view and a longitudinal view of the vessel and an indication of locations of calcium reverb relative to a frame depicted in the cross-sectional view.

### CROSS-REFERENCE TO RELATED APPLICATION

This application claims the benefit of U.S. Patent Application Serial No. 63/753,858, filed February 5, 2025, entitled " CALCIUM FRACTURE INDICATION FOR INTRAVASCULAR IMAGING GRAPHICAL USER INTERFACE”, which is incorporated by reference herein in its entirety.

### TECHNICAL FIELD

The present disclosure generally relates to intravascular imaging systems, such as intravascular ultrasound (IVUS). Particularly, but not exclusively, the present disclosure relates to an improved graphical user interface for intravascular imaging systems comprising an indication of calcium reverb.

### BACKGROUND

Intravascular imaging (IVI) devices are insertable into a patient's vasculature and configured to capture images from within the vessel lumen. Two common intravascular imaging modalities are intravascular ultrasound (IVUS) and optical coherence tomography (OCT). Such imaging modalities have proven diagnostic capabilities for a variety of diseases and disorders. For example, IVI devices are often used as part of a percutaneous coronary intervention (PCI) and can be used to plan, treat, and assess treatment for various obstructive coronary artery diseases, such as, unstable angina, acute myocardial infarction (MI), coronary artery disease (CAD), or the like.

As noted, an example IVI system is an IVUS system. An IVUS system includes a control module with a pulse generator, a motor drive unit, image acquisition and processing components, and a monitor. The IVUS system further includes a catheter with an ultrasound transducer included as part of the distal end of the catheter. The catheter is positioned in a lumen or cavity within, or in proximity to, a region to be imaged, such as a cardiac vessel. Often, a series of images or series of image frames are captured while the catheter is moved (e.g., pulled proximally, or the like) within the vessel lumen.

This series of image frames represent the vessel lumen structure (e.g., vessel wall, lumen, plaque, calcium, stents, etc.) along a longitudinal section of the vessel. Such images can be captured as part of a pre-PCI procedure to aid a physician in determining how to treat the patient, for example, what stent size is appropriate to treat a stenosis, stent landing zones, or the like. Further, such images can be captured as part of a post-PCI procedure to assess the results of the procedure, such as the placement and/or expansion of the stent.

However, it can be difficult for physicians to visualize the complete structure of the vessel lumen and/or the effectiveness of the PCI from the raw series of image frames. For example, with current IVI systems, the physician must identify calcium and the presence of IVUS (or calcium) reverberation from the raw IVI images (e.g., IVUS run, or the like). IVUS calcium reverberation is associated to the thickness of calcium. The physician uses this information to make their clinical decisions. However, as this process is manual, it increases procedure lengths and is a potential area where misinterpretation of calcium within the vessel can occur.

Thus, there is a need for an identification and indication of calcium reverb.

### BRIEF SUMMARY

This Summary is provided to introduce a selection of concepts in a simplified form that are further described below in the Detailed Description. This Summary is not intended to necessarily identify key features or essential features of the claimed subject matter, nor is it intended as an aid in determining the scope of the claimed subject matter.

In general, the present disclosure provides systems and techniques to detect calcium and IVUS reverberation (referred to as calcium reverberation or calcium reverb) from a series of image frames captured via an IVI modality and to generate a graphical user interface (GUI) where the calcium reverb is indicated.

For example, the present disclosure provides an improvement to computing devices and particularly to IVI guidance systems (e.g., an IVUS guidance system, or the like) in that the IVI guidance system provides a graphical user interface arranged to convey calcium reverb and locations of the calcium reverb along the imaged length of the vessel. The examples described herein use an IVUS guidance system as the basis of the examples. However, other IVI modalities could be implemented (e.g., OCT, or the like).

With some embodiments, an IVUS guidance system may include machine learning features to process and analyze the signals generated during an IVUS run. Such information can include automatic detection of calcium, calcium arc length, areas of calcium fracture, and/or calcium reverb within a vessel. The system can be configured to generate a GUI that includes indications of the calcium reverb and display the GUI on a display device. Thereby relieving the physician of manually trying to identify the calcium and calcium reverb locations.

### BRIEF DESCRIPTION OF THE SEVERAL VIEWS OF THE DRAWINGS

To easily identify the discussion of any element or act, the most significant digit or digits in a reference number refer to the figure number in which that element is first introduced.

FIG. 1A illustrates an example cardiac vasculature structure with a stent placed in one of the cardiac arteries and an IVUS catheter inserted into the one of the cardiac arteries.

FIG. 1B illustrates an IVUS imaging system including the IVUS catheter shown in FIG. 1A.

FIG. 2A illustrates an example series of IVUS image frames.

FIG. 2B illustrates a longitudinal representation of the example series of IVUS image frames of FIG. 2A.

FIG. 3 illustrates an IVUS navigation system.

FIG. 4 illustrates a first example graphical user interface (GUI).

FIG. 5A illustrates a second example GUI.

FIG. 5B illustrates a third example GUI.

FIG. 5C illustrates a fourth example GUI.

FIG. 5D illustrates a fifth example GUI.

FIG. 6 illustrates a sixth example GUI.

FIG. 7 illustrates a seventh example GUI.

FIG. 8 illustrates an eighth example GUI.

FIG. 9 illustrates a ninth example GUI.

FIG. 10 illustrates a logic flow for generating a GUI comprising indications of calcium reverb.

FIG. 11 illustrates a computer-readable storage medium.

FIG. 12 illustrates a diagrammatic representation of a machine.

### DETAILED DESCRIPTION

The foregoing has broadly outlined the features and technical advantages of the present disclosure such that the following detailed description of the disclosure may be better understood. It is to be appreciated by those skilled in the art that the embodiments disclosed may be readily utilized as a basis for modifying or designing other structures for carrying out the same purposes of the present disclosure. The novel features of the disclosure, both as to its organization and operation, together with further objects and advantages will be better understood from the following description when considered in connection with the accompanying figures. It is to be expressly understood, however, that each of the figures is provided for the purpose of illustration and description and is not intended as a definition of the limits of the present disclosure.

As noted, the present disclosure relates to IVI guidance systems, such as, IVUS guidance systems. Further, the present disclosure relates to automatic assessment of the IVI images. In particular, the disclosure provides a graphical user interface (GUI) arranged to convey information related to the IVI images and assessment of the vessel and/or lesion.

As noted, the disclosure uses IVUS as the example imaging modality. As such, an example IVUS imaging system, cardiac vasculature structure, and series of IVUS images are described prior to describing the improvements to IVI guidance systems and the GUI of the present disclosure.

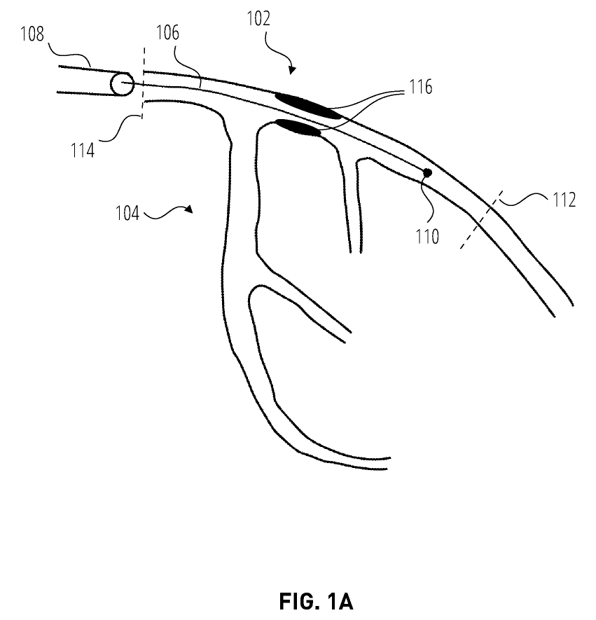

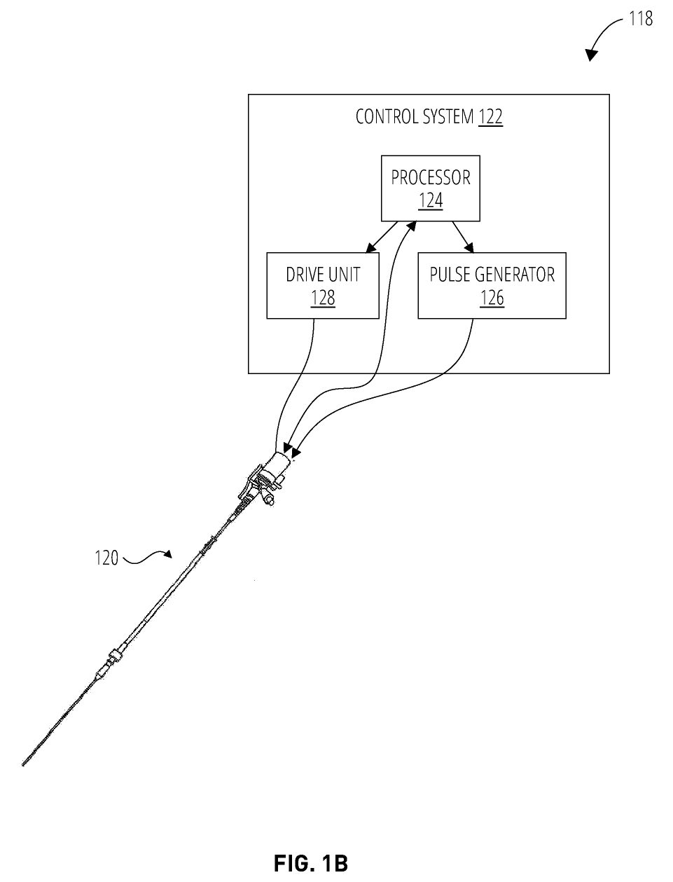

FIG. 1A illustrates a cardiac vasculature structure 100 having a cardiac artery 102 (e.g., cardiac artery, vessel, or the like) with several side branches 104 (e.g., descending arteries, marginal arteries, vessels, or the like) branching off the cardiac artery 102.

It is to be appreciated that various cardiac interventions, often referred to as percutaneous coronary intervention (PCI). can be guided by IVUS.

For example, prior to performing a PCI procedure, during a PCI procedure, and/or after a PCI procedure; an intravascular imaging catheter (e.g., IVUS imaging catheter 106) is inserted into the cardiac artery 102 via a guide catheter 108 and images of the cardiac artery 102 are captured. These images are often captured pre-PCI as well as post-PCI. During imaging, the IVUS imaging catheter 106 includes an ultrasound transducer 110 disposed on a distal end of the IVUS imaging catheter 106. The IVUS imaging catheter 106 can be pulled back from a distal point 112 in the cardiac artery 102 to a proximal point 114 in the cardiac artery 102 and intravascular image frames are captured while the IVUS imaging catheter 106 is pulled back.

As indicated in this figure, the cardiac artery 102 includes calcium 116 disposed in the vessel. Often, in a PCI procedure, a physician can choose to break up calcium in the cardiac artery 102 (e.g., the calcium 116, or the like). It is to be appreciated that by breaking up the calcium or causing a calcium “fracture” the efficacy or outcome is the PCI procedure is improved. For example, fractured calcium allows for better expansion (e.g., stent expansion, or the like). Further, the thickness of the calcium affects calcium fracture treatment selection as well as overall treatment outcomes.

FIG. 1B illustrates an IVUS imaging system 118. The IVUS imaging system 118 is depicted including IVUS imaging catheter 106, which is couplable to a control system 122. The control system 122 may include, for example, processor 124, pulse generator 126, and drive unit 128. In at least some embodiments, the pulse generator 126 forms electric pulses that may be input to one or more transducers (e.g., ultrasound transducer 110, or the like) disposed on IVUS imaging catheter 106.

With some embodiments, ultrasound transducer 110 can be part of an imaging core and coupled to the proximal end of IVUS imaging catheter 106 via a drive cable or drive shaft. Mechanical energy from the drive unit 128 can be used to rotate the imaging core (and thus the ultrasound transducer 110).

The electrical pulses generated by pulse generator 126 can be delivered to the ultrasound transducer 110 while the ultrasound transducer 110 is rotated by the drive unit 128. The electrical signals are transformed by the ultrasound transducer 110 into acoustic pulses that are transmitted through the tissue of cardiac vasculature structure 100. The tissue and/or structure (e.g., calcium, etc.) of the cardiac vasculature structure 100 reflects the acoustic pulses, which are absorbed by the ultrasound transducer 110 and transformed into electric pulses. The transformed electric pulses are delivered to the processor 124 and converted into IVUS images displayable on a monitor.

In general, IVUS imaging system 118 can detect calcium 116 from images of the cardiac artery 102 captured by the IVUS imaging catheter 106. The calcium, and particularly the locations and calcium reverb, can be identified. For example, processor 124 can implement a calcium detection algorithm. Further, processor 124 can generate a GUI comprising indications of the detected calcium and locations and calcium reverb along the cardiac artery 102. As such, the GUI provided herein will facilitate quick identifications of the presence of calcium reverb within the intravascular ultrasound image, thereby increasing workflow, outcomes, and patient safety.

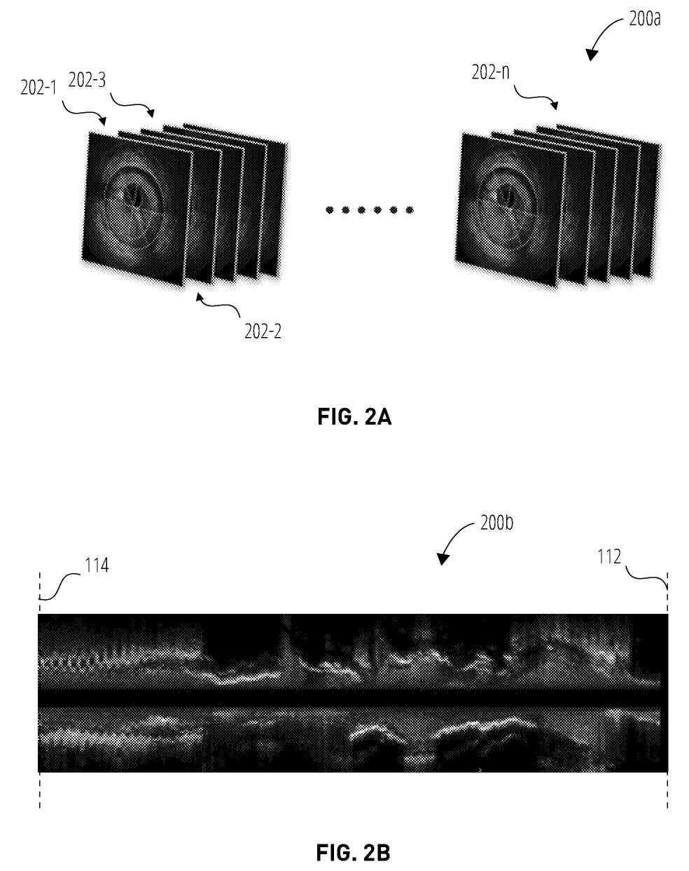

FIGS. 2A and 2B illustrate a series of IVUS image frames 200a and a longitudinal view 200b of the series of IVUS image frames 200a. As described above, several IVUS image frames can be captured while IVUS imaging catheter 108 is moved through cardiac artery 102. For example, FIG. 2A depicts the series of IVUS image frames 200a including IVUS image frames 202-1, 202-2, 202-3 to 202-n, where n is a positive integer, in this case, greater than ten (10). In practice, n can be any positive integer greater than or equal to two (2) but will often be greater than one hundred.

The series of IVUS image frames 200a can be stacked or grouped and depicted longitudinally to represent a longitudinal slice of the cardiac artery 102 from distal point 114 to proximal point 116. For example, FIG. 2B illustrates longitudinal view 200b which shows a longitudinal slice of cardiac artery 102 from distal point 114 to proximal point 116 formed from the series of IVUS image frames 200a.

As noted above, calcium and/or calcium reverb may be represented in the series of IVUS image frames 200a. The disclosure provides to detect calcium reverb from the series of IVUS image frames 200a and to generate a GUI comprising indications of the location of the detected calcium reverb.

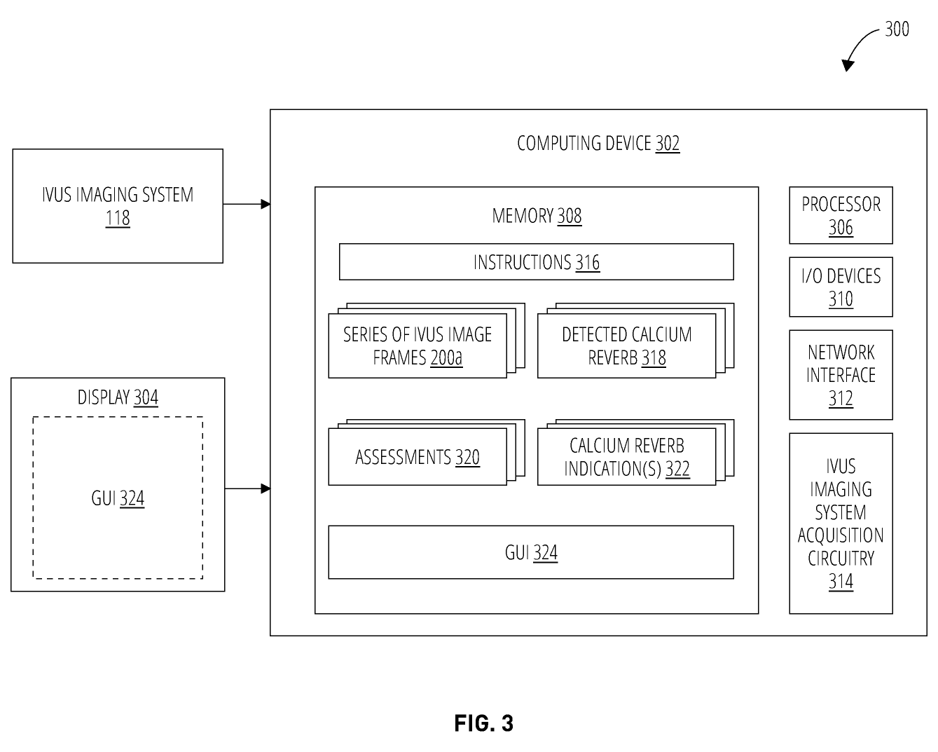

FIG. 3 illustrates an IVUS image visualization system 300, which can be implemented to detect calcium and calcium reverb and display the locations of the detected calcium reverb as part of a pre-PCI procedure. In general, IVUS image visualization system 300 is a system for processing, annotating, and/or presenting IVUS images. IVUS image visualization system 300 can be implemented in a commercial IVUS guidance or navigation system, such as, for example, the AVVIGO^{TM} Guidance System available from Boston Scientific®. The present disclosure provides advantages over prior or conventional IVUS navigation systems in that the locations of calcium reverb are identified, thereby allowing a user (e.g., physician, or the like) to select relevant locations and treatment options and make clinical decisions to improve patient outcomes in a post-PCI procedure.

IVUS image visualization system 300 includes a computing device 302 and display 304. Optionally, IVUS image visualization system 300 includes an IVUS imaging system, such as IVUS imaging system 118. Where IVUS image visualization system 300 includes IVUS imaging system 118, IVUS image visualization system 300 could be implemented as part of control system 122 or alternatively, control system 122 could be implemented as part of computing device 302 of IVUS image visualization system 300. IVUS image visualization system 300 will be described with reference to cardiac vasculature structure 100 and IVUS imaging system 118 of FIGS. 1A and 1B as well as the series of IVUS image frames 200a and longitudinal view 200b of FIGS. 2A and 2B, for clarity and ease of discussion. However, it is noted that IVUS image visualization system 300 could be implemented for use with another IVUS imaging system or even another intravascular imaging system utilizing a different imaging modality than IVUS imaging system 118.

Computing device 302 can be any of a variety of computing devices. In some embodiments, as noted above, computing device 302 can be incorporated into and/or implemented by a console to be coupled to an intravascular imaging device (e.g., IVUS imaging system 118, IVUS imaging catheter 106, or the like). With some embodiments, computing device 302 can be a workstation or server communicatively coupled to IVUS imaging system 118. With still other embodiments, computing device 302 can be provided by a cloud-based computing device, such as, by a computing as a service system accessibly over a network (e.g., the Internet, an intranet, a wide area network, or the like). Computing device 302 can include processor 306, memory 308, input and/or output (I/O) devices 310, network interface 312, and IVUS imaging system acquisition circuitry 314.

Display 304 may include any of a variety of devices arranged to display graphical information, such as, a light emitting diode (LED) display, or the like. It is to be appreciated that although display 304 is depicted separate from computing device 302, display 304 could be implemented as part of computing device 302 or be distinct from computing device 302.

The processor 306 may include circuity or processor logic, such as, for example, any of a variety of commercial processors. In some examples, processor 306 may include multiple processors, a multi-threaded processor, a multi-core processor (whether the multiple cores coexist on the same or separate dies), and/or a multi-processor architecture of some other variety by which multiple physically separate processors are in some way linked. Additionally, in some examples, the processor 306 may include graphics processing portions and may include dedicated memory, multiple-threaded processing and/or some other parallel processing capability. In some examples, the processor 306 may be an application specific integrated circuit (ASIC) or a field programmable integrated circuit (FPGA).

The memory 308 may include logic, a portion of which includes arrays of integrated circuits, forming non-volatile memory to persistently store data or a combination of non-volatile memory and volatile memory. It is to be appreciated, that the memory 308 may be based on any of a variety of technologies. In particular, the arrays of integrated circuits included in memory 308 may be arranged to form one or more types of memory, such as, for example, dynamic random-access memory (DRAM), NAND memory, NOR memory, or the like.

I/O devices 310 can be any of a variety of devices to receive input and/or provide output. For example, I/O devices 310 can include, a keyboard, a mouse, a joystick, a foot pedal, a display, a touch enabled display, a haptic feedback device, an LED, or the like.

Network interface 312 can include logic and/or features to support a communication interface. For example, network interface 312 may include one or more interfaces that operate according to various communication protocols or standards to communicate over direct or network communication links. Direct communications may occur via use of communication protocols or standards described in one or more industry standards (including progenies and variants). For example, network interface 312 may facilitate communication over a bus, such as, for example, peripheral component interconnect express (PCIe), non-volatile memory express (NVMe), universal serial bus (USB), system management bus (SMBus), SAS (e.g., serial attached small computer system interface (SCSI)) interfaces, serial AT attachment (SATA) interfaces, or the like. Additionally, network interface 312 can include logic and/or features to enable communication over a variety of wired or wireless network standards. For example, network interface 312 may be arranged to support wired communication protocols or standards, such as, Ethernet, or the like. As another example, network interface 312 may be arranged to support wireless communication protocols or standards, such as, for example, Wi-Fi, Bluetooth, 5G, or the like.

The IVUS imaging system acquisition circuitry 314 may include circuity including custom manufactured or specially programmed circuitry configured to receive or receive and send signals with IVUS imaging system 118, including indications of intravascular images, intravascular image frames, or a series of intravascular image frames. For example, IVUS imaging system acquisition circuitry 314 can include image acquisition circuitry (not shown) and/or pulse generator 126.

Memory 308 can include instructions 316, series of IVUS image frames 200a, detected calcium reverb 318, assessments 320, calcium reverb indications 322, and GUI 324.

During operation, processor 306 can execute instructions 316 to cause computing device 302 to receive (e.g., from IVUS imaging system 118, or the like) a recording of an “IVUS run” and store the recording as the series of IVUS image frames 200a in memory 308. For example, processor 306 can execute instructions 316 to receive information elements from IVUS imaging system 118 comprising indications of IVUS image frames 202-1, 202-2, 202-3 to 202-n, captured by IVUS imaging catheter 106 while IVUS imaging catheter 106 is pulled through cardiac artery 102 from distal point 112 to proximal point 114 through the calcium 116. It is to be appreciated that the series of IVUS image frames 200a can be stored in a variety of image formats or even non-image formats or data structures.

The present disclosure provides to process the series of IVUS image frames 200a to detect calcium and calcium reverb from the series of IVUS image frames 200a and display visual indications of the locations of the detected calcium reverb via a GUI. In some examples, processor 306 can execute instructions 316 to receive indications of calcium reverb from series of IVUS image frames 200a and store the indications as detected calcium reverb 318. With some examples, processor 306 can execute instructions 316 to detect the calcium reverb represented in the series of IVUS image frames 200a and store indications of the detected calcium reverb as detected calcium reverb 318. For example, processor 306 can execute instructions 316 to identify frames of series of IVUS image frames 200a (e.g., IVUS image frames 202-2, 202-3, etc.) where calcium is detected and identify areas of IVUS reverberation indicative of calcium thickness in the calcium and store indications of the calcium reverb and/or the frames of the series of IVUS image frames 200a associated with the detected calcium reverb as detected calcium reverb 318.

Complete details of calcium reverb detection techniques, such as techniques to identify frames of series of IVUS image frames 200a in which calcium is present is beyond the scope of this disclosure. However, with some examples, processor 306 can execute instructions 316 to identify the detected calcium reverb 318 from the series of IVUS image frames 200a using machine learning (ML) models trained to identify frames of a series of frames in which calcium is represented. As another example, processor 306 can execute instructions 316 to identify the detected calcium reverb 318 from the series of IVUS image frames 200a using image processing algorithms (e.g., image segmentation, etc.). With yet another example, processor 306 can execute instructions 316 to identify the detected calcium reverb 318 from the series of IVUS image frames 200a using a combination of image processing techniques and ML models. For example, processor 306 can execute instructions 316 to apply a cross-sectional segmentation to the frames of the series of IVUS image frames 200a and then infer frames representing calcium reverb from the frame segmentations using an ML model.

Further, processor 306 can execute instructions 316 to derive the assessments 320 for each of the detected calcium reverb 318. In general, for each detected calcium reverb 318, the assessments 320 can include vessel and/or lumen borders, raw vessel and/or lumen areas, smoothed vessel and/or lumen areas, a plaque burden, calcium arc length, calcium reverb present, or the like. Complete detail of derivation of stent assessments is beyond the scope of this disclosure. However, with some examples, processor 306 can execute instructions 316 to determine the assessments (e.g., boundaries, area, plaque burden, expansion ratio, etc.) of the vessel and/or lumen depicted in each frame of the series of IVUS image frames 200a associated with each detected calcium reverb 318. In some embodiments, this can include identifying key frames and then deriving the assessments 320 based on the identified key frames. With some embodiments, processor 306 can execute instructions 316 to determine the assessments 320 using image processing techniques, ML models, or a combination of image processing techniques and ML models.

Examples of processing IVUS images to detect calcium may be described in more detail in United States Patent Application Publication No. 2023/0157672, titled “Intravascular Ultrasound Imaging and Calcium Detection Methods” and filed on March 1, 2022; United States Patent Application Publication No. 2023/0112017, titled “Medical Device Systems for Automatic Lesion Assessment” and filed on October 5, 2022; United States Patent Application Publication No. 2023/0380806, titled “Systems and Methods for Intravascular Visualization” and filed on May 26, 2023; United States Patent Application Publication No. 2024/0081781, which applications are each incorporated herein by reference in their entirety.

Processor 306 can execute instructions 316 to generate calcium reverb indication 322 (or indications) for detected calcium reverb 318 and to generate GUI 324 from the calcium reverb indications 322. In general, the calcium reverb indications 322 and/or GUI 324 can comprise visual indications of the series of IVUS image frames 200a, the detected calcium reverb 318, and the assessments 320. The areas with detected calcium reverb 318 and/or assessments 320 can be visually depicted in GUI 324.

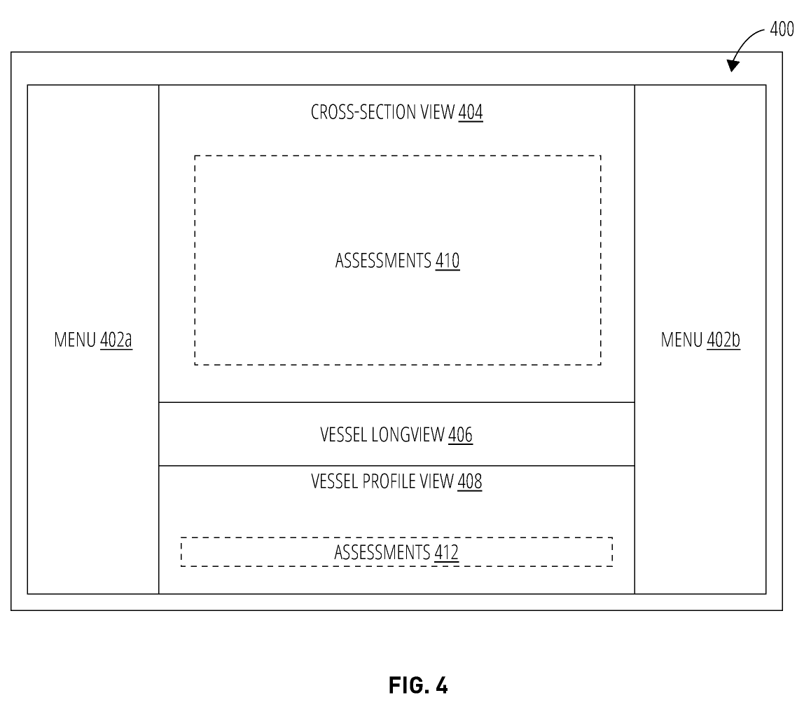

FIG. 4 illustrates a GUI 400, which can be generated according to some embodiments of the present disclosure. In some embodiments, GUI 400 can be generated by IVUS image visualization system 300 as GUI 324 and displayed on display 304. As depicted, GUI 400 includes several graphical components or visual indications, such as, menus 402a and 402b, cross-section view 404, and vessel long view 406, and vessel profile view 408.

With some embodiments, a processor (e.g., processor 124, processor 306, or the like) can be configured to execute instructions (e.g., instructions 316, or the like) to generate GUI 400. In some examples, GUI 400 can be generated responsive to an automatic calcium and/or calcium reverb detection process. For example, with some embodiments, IVUS image visualization system 300 can be arranged to automatically detect calcium in cardiac artery 102 from the series of IVUS image frames 200a and identify locations of calcium reverb (e.g., via machine learning, image classification, or the like). Responsive to this detection, GUI 400 could be generating to include visual indications of the locations of the calcium reverb.

Menu 402a can comprise GUI inputs such as button, drop down menus, selection icons, or the like. Menu 402a can include GUI input options to select measurement and annotation tools, length tools, modification reset buttons, or the like. Menu 402b can comprise GUI inputs such as buttons, drop down menus, selection icons, or the like. Menu 402b can include GUI inputs options to select views related to views of the IVUS images, layout options, annotations, navigation, dynamic review options, status of the computing device, or the like.

Cross-section view 404 can comprise a cross-sectional view, or on-axis view, of one (e.g., a frame, or the like) of the series of IVUS image frames 200a. For example, cross-section view 404 can include IVUS image frame 202-1 (or IVUS image frame 202-2 to 202-n). Further, cross-section view 404 can include assessments 410. With some examples, the assessments can include visual indications of vessel and/or lumen borders, indications of vessel and/or lumen area, indications of a plaque burden, indications of a calcium arc length, and/or a calcium reverb indication 322.

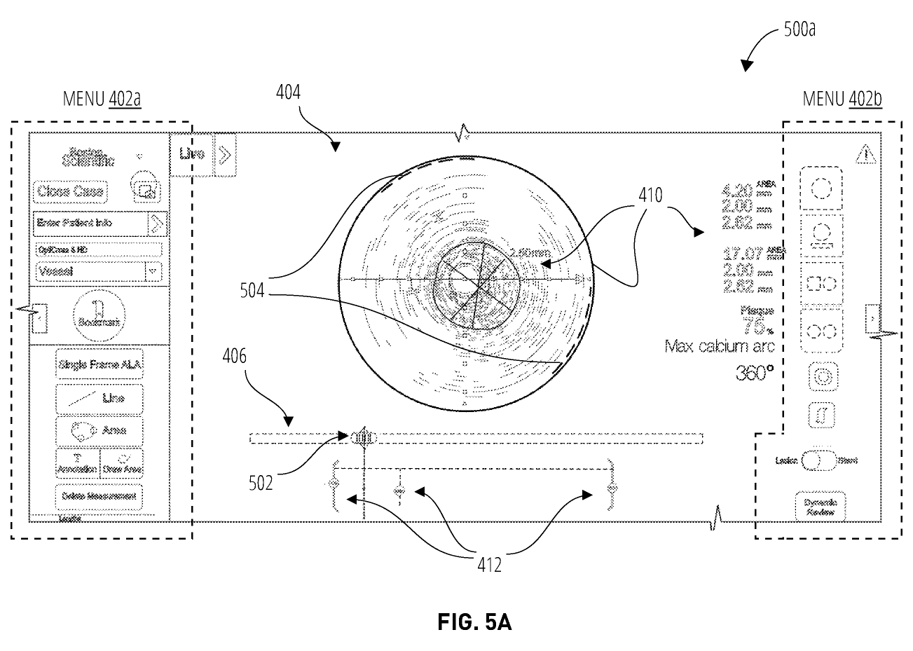

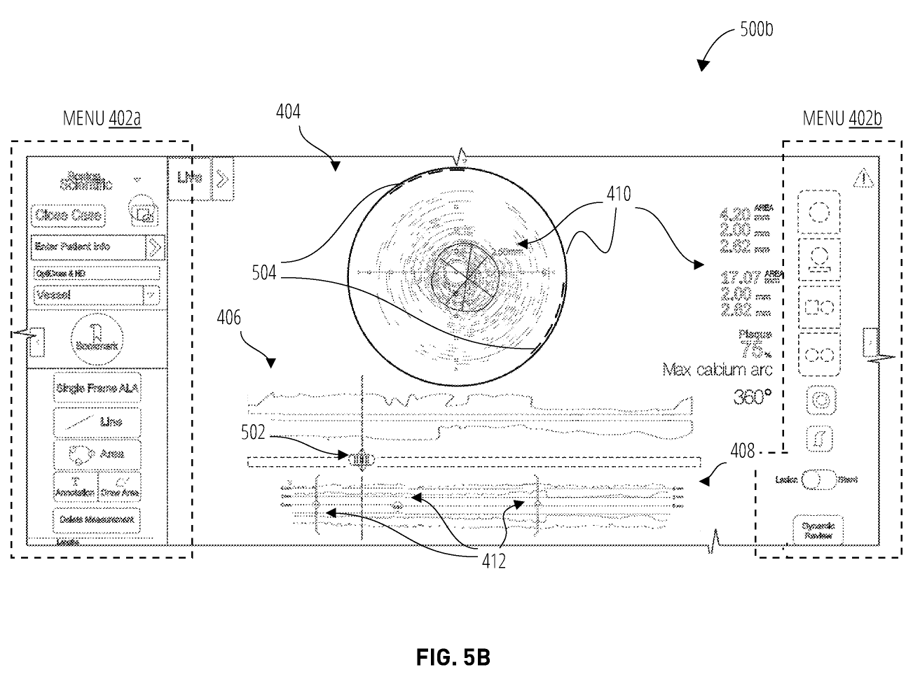

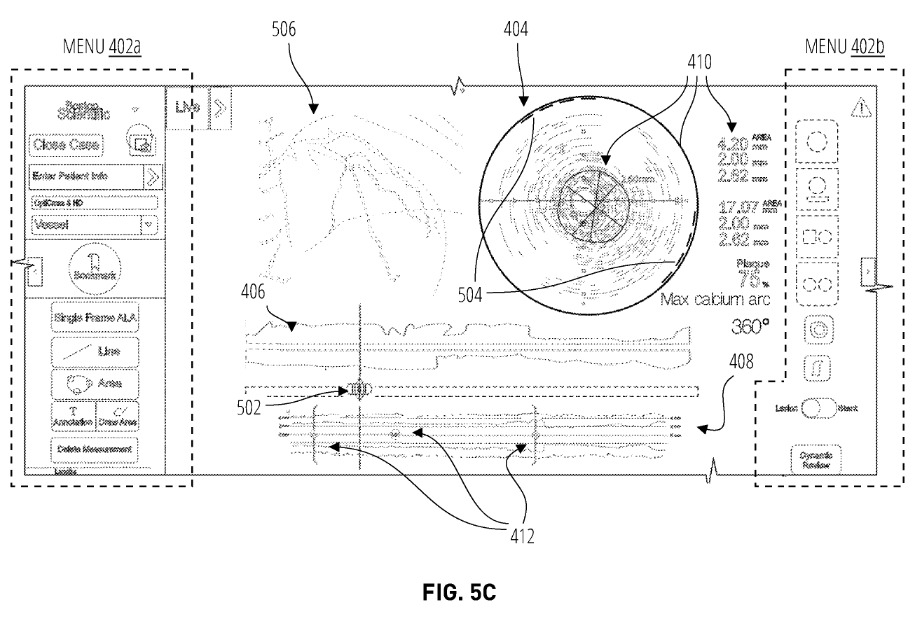

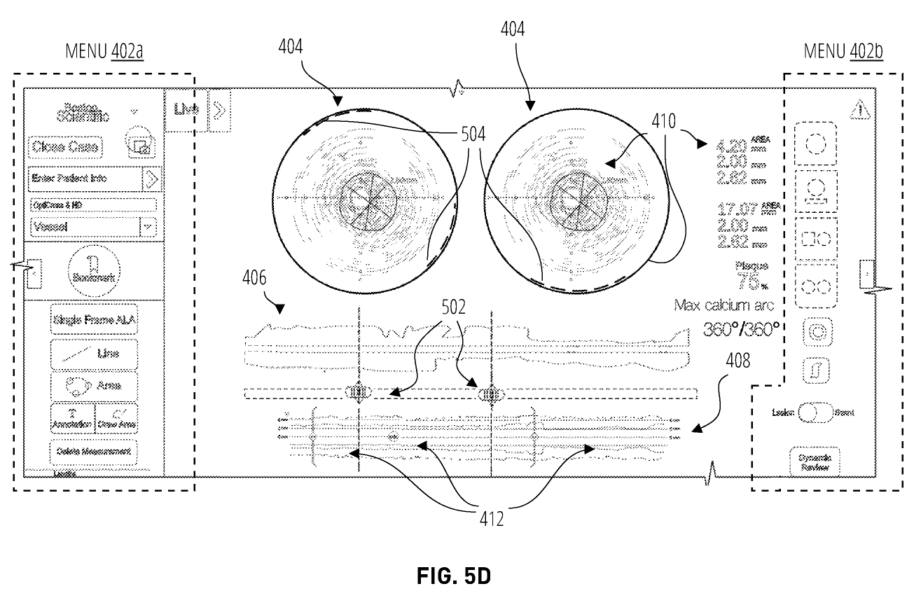

Vessel long view 406 can include the series of IVUS image frames 200a represented longitudinally, for example, as depicted in longitudinal view 200b of FIG. 2B. Vessel profile view 408 can include a visual depiction of the longitudinal profile of the vessel. For example, vessel profile view 408 can include indications of the lumen border and vessel border, indications of plaque, indications of a stent, indications of key frames, or the like. Further, vessel long view 406 and vessel profile view 408 can include a navigation slider (see FIG. 5A to FIG. 5D), which can be selected and used by a user (e.g., physician, or the like) to navigate through the series of IVUS image frames 200a. In some examples, the slider can be linked to the cross-section view 404 such that the frame depicted in cross-section view 404 corresponds to the location of the slider along longitudinal view 200b. That is, as the slider is moved the image displayed in cross-section view 404 can change to match the location of the slider.

Further, vessel long view 406 and vessel profile view 408 can include assessments 412. With some examples, assessments 412 can include visual indications of plaque, plaque burden, calcium, detected calcium reverb 318, stent expansion, or the like.

FIG. 5A illustrates a GUI, which can be generated according to some embodiments of the present disclosure. For example, GUI 500a can be generated by IVUS image visualization system 300 as GUI 324 and displayed on display 304. With some embodiments, responsive to receiving the series of IVUS image frames 200a, processor 306 can execute instructions 316 to detect calcium 116 and generate detected calcium reverbs 318. Further, processor 306 can execute instructions 316 to generate GUI 500a comprising visual indications of the detected calcium reverb 318.

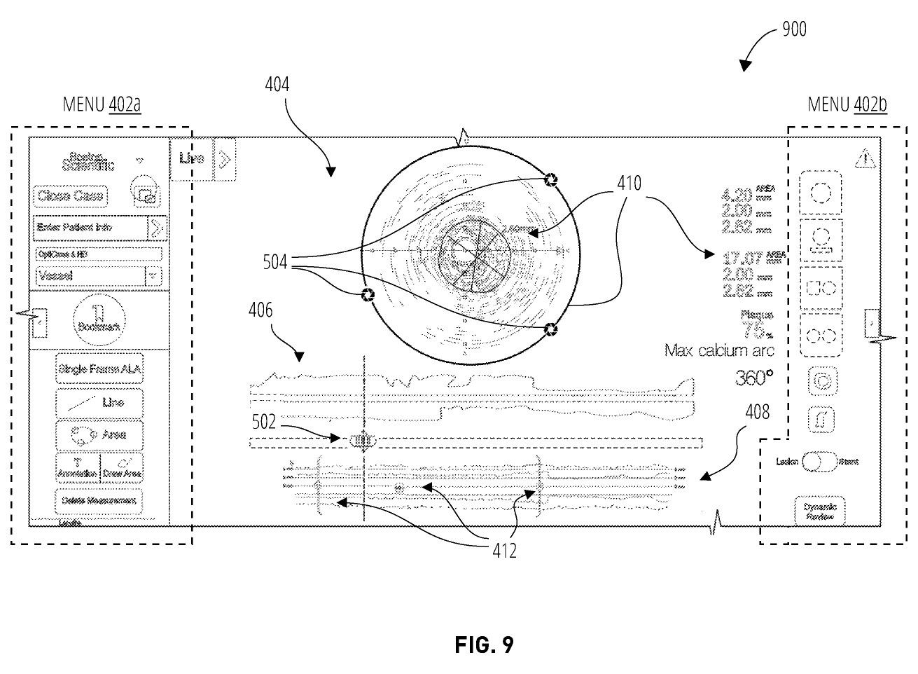

GUI 500a can include menus 402a and 402b, cross-section view 404 and vessel long view 406. In vessel long view 406, slider 502 is represented. As described above, GUI 500a provides slider 502 such that a user can scrub across the series of IVUS image frames 200a. Further, in cross-section view 404 a frame of the series of IVUS image frames 200a (e.g., corresponding to the slider location, or the like) is depicted along with various assessments 410 (e.g., lumen and vessel borders, area, plaque burden, calcium arc, etc. Further, calcium reverb icon 504 is depicted in a location around the radius of the vessel showing the area of calcium reverb. With some examples, the calcium reverb icon can be features lines (e.g., dashed, shadowed, colored, etc.) indicating areas of detected calcium reverb. With other examples (e.g., FIG. 9) the calcium reverb icon can be a dark circle (e.g., black) with a lighter colored (e.g., white) central geometric feature (e.g., octagon, or the like) with lines emanating from the central geometric feature. It is noted that multiple areas of reverb could be shown (FIG. 9) for one frame of series of IVUS image frames 200a.

Additionally, GUI 500a can include assessments 412 in vessel long view 406. With some examples, assessments 412 can include visual indications of key frames (e.g., brackets, or the like), a visual indication of the location of the minimum lumen area, etc.

FIG. 5B illustrates a GUI 500b, which can be generated according to some embodiments of the present disclosure. For example, GUI 500b can be generated by IVUS image visualization system 300 as GUI 324 and displayed on display 304. With some embodiments, responsive to receiving the series of IVUS image frames 200a, processor 306 can execute instructions 316 to detect calcium 116 and generate detected calcium reverbs 318. Further, processor 306 can execute instructions 316 to generate GUI 500b comprising visual indications of the detected calcium reverb 318.

Like GUI 500a, GUI 500b can include menus 402a and 402b, cross-section view 404 and vessel long view 406. Further, GUI 500b includes vessel profile view 408 and the vessel long view 406 depicts longitudinal view 200b. GUI 500b likewise includes slider 502, a frame of the series of IVUS image frames 200a depicted in the cross-section view 404 and the vessel profile view 408 depicted below the vessel long view 406. Likewise, GUI 500b includes assessments 410, assessments 412, and calcium reverb icon 504. Like depicted in GUI 500a, the calcium reverb icon 504 of GUI 500b is depicted in a location around the radius of the vessel showing the area of calcium reverb.

FIG. 5C illustrates a GUI 500c, which can be generated according to some embodiments of the present disclosure. For example, GUI 500c can be generated by IVUS image visualization system 300 as GUI 324 and displayed on display 304. With some embodiments, responsive to receiving the series of IVUS image frames 200a, processor 306 can execute instructions 316 to detect calcium 116 and generate detected calcium reverbs 318. Further, processor 306 can execute instructions 316 to generate GUI 500c comprising visual indications of the detected calcium reverb 318.

Like GUIs 500a and 500b, GUI 500c can include menus 402a and 402b, cross-section view 404, vessel long view 406, and vessel profile view 408. Further, GUI 500c includes a co-registration view 506, which depicts an extravascular image of the vessel represented in series of IVUS image frames 200a. With some examples, the co-registration view 506 can include visual depictions of locations along the longitudinal view 200b (e.g., key frames, etc.). GUI 500c likewise includes slider 502, a frame of the series of IVUS image frames 200a depicted in the cross-section view 404 and the vessel profile view 408 depicted below the vessel long view 406. Likewise, GUI 500c includes assessments 410, assessments 412, and calcium reverb icon 504. Like depicted in GUIs 500a and 500b, the calcium reverb icon 504 of GUI 500c is depicted in a location around the radius of the vessel showing the area of calcium reverb.

FIG. 5D illustrates a GUI 500d, which can be generated according to some embodiments of the present disclosure. For example, GUI 500d can be generated by IVUS image visualization system 300 as GUI 324 and displayed on display 304. With some embodiments, responsive to receiving the series of IVUS image frames 200a, processor 306 can execute instructions 316 to detect calcium 116 and generate detected calcium reverbs 318. Further, processor 306 can execute instructions 316 to generate GUI 500d comprising visual indications of the detected calcium reverb 318.

Like GUIs 500a, 500b and 500c, GUI 500d can include menus 402a and 402b, vessel long view 406 and vessel profile view 408. Further, GUI 500d includes two cross-section views 404 and two sliders 502 where the frames of series of IVUS image frames 200a depicted in each respective cross-section view 404 correspond to the locations of a respective one of sliders 502. GUI 500d likewise includes the vessel profile view 408 depicted below the vessel long view 406, assessments 410, assessments 412, and calcium reverb icon 504. Like depicted in GUIs 500a, 500b and 500c, the calcium reverb icon 504 of GUI 500c is depicted in a location around the radius of the vessel showing the area of calcium reverb.

It is noted that multiple calcium reverb icons 504 per cross-section view 404 could be depicted (e.g., where multiple reverb locations are detected, or the like). Or only one of the 404 could depict calcium reverb icons 504 (e.g., where no calcium reverb is detected in the respective frame).

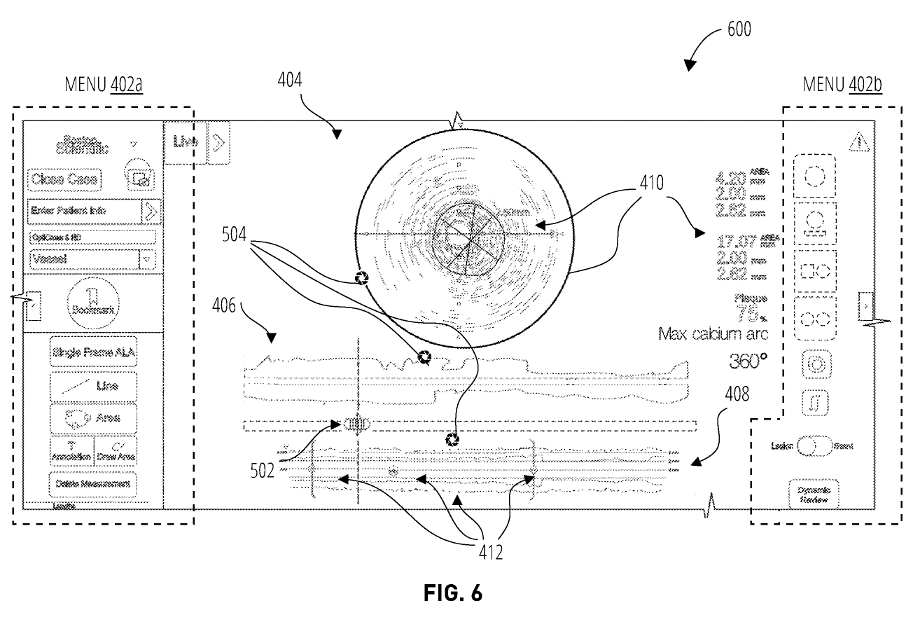

FIG. 6 illustrates a GUI 600, which can be generated according to some embodiments of the present disclosure. For example, GUI 600 can be generated by IVUS image visualization system 300 as GUI 324 and displayed on display 304. With some embodiments, responsive to receiving the series of IVUS image frames 200a, processor 306 can execute instructions 316 to detect calcium 116 and generate detected calcium reverbs 318. Further, processor 306 can execute instructions 316 to generate GUI 600 comprising visual indications of the detected calcium reverb 318.

GUI 600 is like GUI 500b but includes multiple calcium reverb icons 504. For example, GUI 600 includes a calcium reverb icon 504 depicted around the radius of the vessel like in GUI 500b. Further, GUI 600 can include a calcium reverb icon 504 depicted on vessel long view 406 and/or vessel profile view 408. In some examples, GUI 600 could include any one or more of the calcium reverb icons 504. Further, GUI 600 includes assessments 412 (e.g., dark solid line on lumen border) in vessel profile view 408 showing the length of detected calcium.

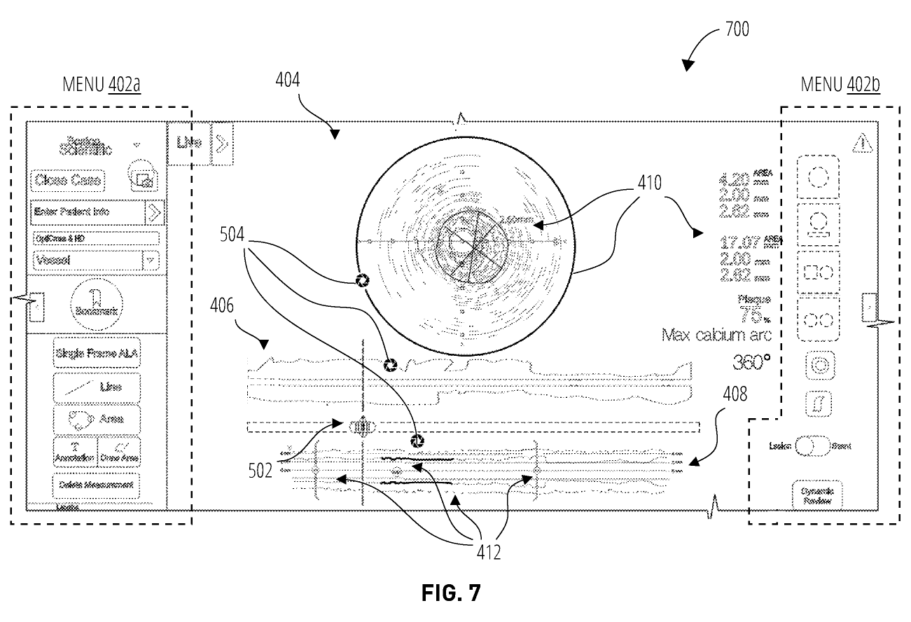

FIG. 7 illustrates a GUI 700, which can be generated according to some embodiments of the present disclosure. For example, GUI 700 can be generated by IVUS image visualization system 300 as GUI 324 and displayed on display 304. With some embodiments, responsive to receiving the series of IVUS image frames 200a, processor 306 can execute instructions 316 to detect calcium 116 and generate detected calcium reverbs 318. Further, processor 306 can execute instructions 316 to generate GUI 700 comprising visual indications of the detected calcium reverb 318.

GUI 700 is like GUI 500b but includes multiple calcium reverb icons 504. For example, GUI 700 includes a calcium reverb icon 504 depicted around the radius of the vessel like in GUI 500b. Further, GUI 700 can include a calcium reverb icon 504 depicted on vessel long view 406 and/or vessel profile view 408. In some examples, GUI 700 could include any one or more of the calcium reverb icons 504. Further, GUI 700 includes assessments 412 (e.g., dark solid line on lumen border) in vessel profile view 408 showing the length of detected calcium.

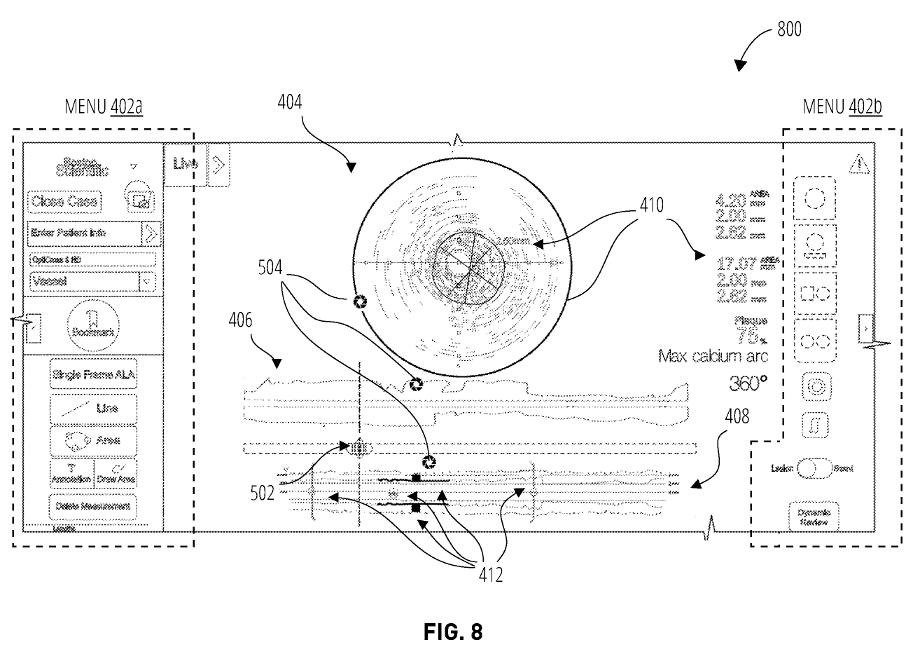

FIG. 8 illustrates a GUI 800, which can be generated according to some embodiments of the present disclosure. For example, GUI 800 can be generated by IVUS image visualization system 300 as GUI 324 and displayed on display 304. With some embodiments, responsive to receiving the series of IVUS image frames 200a, processor 306 can execute instructions 316 to detect calcium 116 and generate detected calcium reverbs 318. Further, processor 306 can execute instructions 316 to generate GUI 800 comprising visual indications of the detected calcium reverb 318.

GUI 800 is like GUI 700 but includes other assessments 412 (e.g., raised solid line on lumen border) in vessel profile view 408 showing areas where the calcium arc is 360°.

FIG. 9 illustrates a GUI 900, which can be generated according to some embodiments of the present disclosure. For example, GUI 900 can be generated by IVUS image visualization system 300 as GUI 324 and displayed on display 304. With some embodiments, responsive to receiving the series of IVUS image frames 200a, processor 306 can execute instructions 316 to detect calcium 116 and generate detected calcium reverbs 318. Further, processor 306 can execute instructions 316 to generate GUI 900 comprising visual indications of the detected calcium reverb 318.

GUI 900 is like GUI 500a but includes multiple calcium reverb icons 504 depicted around the radius of the vessel, showing areas of calcium reverb. Further, the calcium reverb icons 504 depicted in FIG. 9 are different from the icons depicted in GUI 500a of FIG. 5A. In some examples, the calcium reverb icons can be mixed and matched (e.g., features lines shown in cross-sectional view 404 and geometric icon shown in longitudinal view 406 or vessel profile view 408).

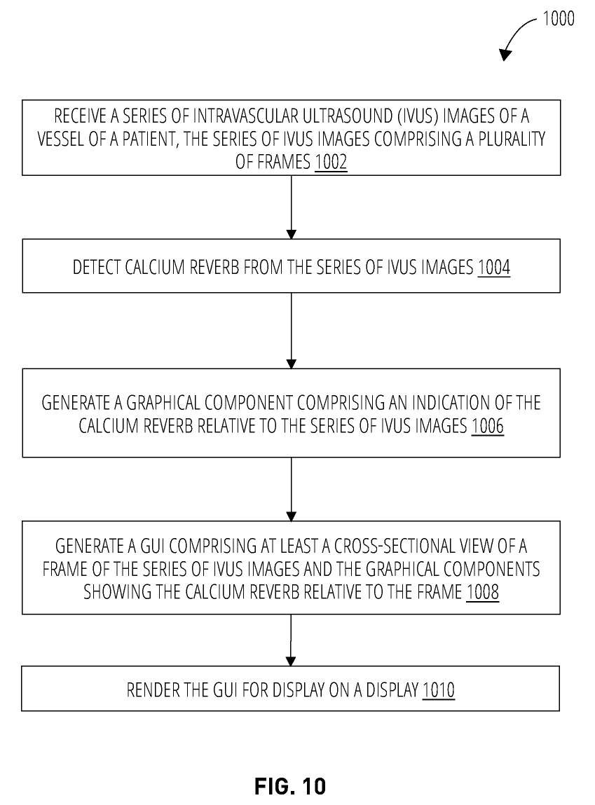

FIG. 10 illustrates a logic flow 1000 to generate a GUI, according to some embodiments of the present disclosure. The logic flow 1000 can be implemented by IVUS image visualization system 300 and will be described with reference to IVUS image visualization system 300 for clarity of presentation. However, it is noted that logic flow 1000 could also be implemented by an IVUS guidance system different than IVUS image visualization system 300.

Logic flow 1000 can begin at block 1002. At block 1002 “receive a series of intravascular ultrasound (IVUS) images of a vessel of a patient, the series of IVUS images comprising a plurality of frames” a series of IVUS images captured via an IVUS catheter percutaneously inserted in a vessel of a patent can be received. For example, information elements comprising indications of the series of IVUS image frames 200a can be received from IVUS imaging system 118 where catheter 120 is (or was) percutaneously inserted into cardiac artery 102. The series of IVUS image frames 200a can comprise frames (e.g., IVUS image frames 202-1 to 202-n) representative of images captured while the catheter 120 is pulled back from the distal point 112 to the proximal point 114. Processor 306 can execute instructions 316 to receive information elements comprising indications of series of IVUS image frames 200a from IVUS imaging system 118, or directly from catheter 120 as may be the case.

Continuing to block 1004 “detect calcium reverb from the series of IVUS images” calcium reverb can be detected from the series of IVUS images. For example, processor 306 can execute instructions 316 to detect (e.g., using machine learning and/or image processor) calcium and calcium reverb in the vessel based on the frames of series of IVUS image frames 200a. Continuing to block 1006 “generate a graphical component comprising an indication of the calcium reverb relative to the series of IVUS images” a graphical component comprising an indication of the calcium reverb is generated. For example, processor 306 can execute instructions 316 to generate assessments 320 comprising a calcium reverb icon.

Continuing to block 1008 “generate a GUI comprising a cross-sectional view of a frame of the series of IVUS images and the graphical component showing the calcium reverb relative to the frame” a GUI comprising a cross-sectional view of a frame of the series of IVUS images and the graphical component indicating the detected calcium reverb relative to the frame is generated. For example, processor 306 can execute instructions 316 to generate GUI 324 comprising cross-section view 404 and indications (e.g., calcium reverb icon 504) of calcium reverb relative to the frame depicted in cross-section view 404. Continuing to block 1010 “render the GUI for display on a display” the GUI can be rendered for display. For example, processor 306 can execute instructions 316 to generate GUI 324 for display by display 304.

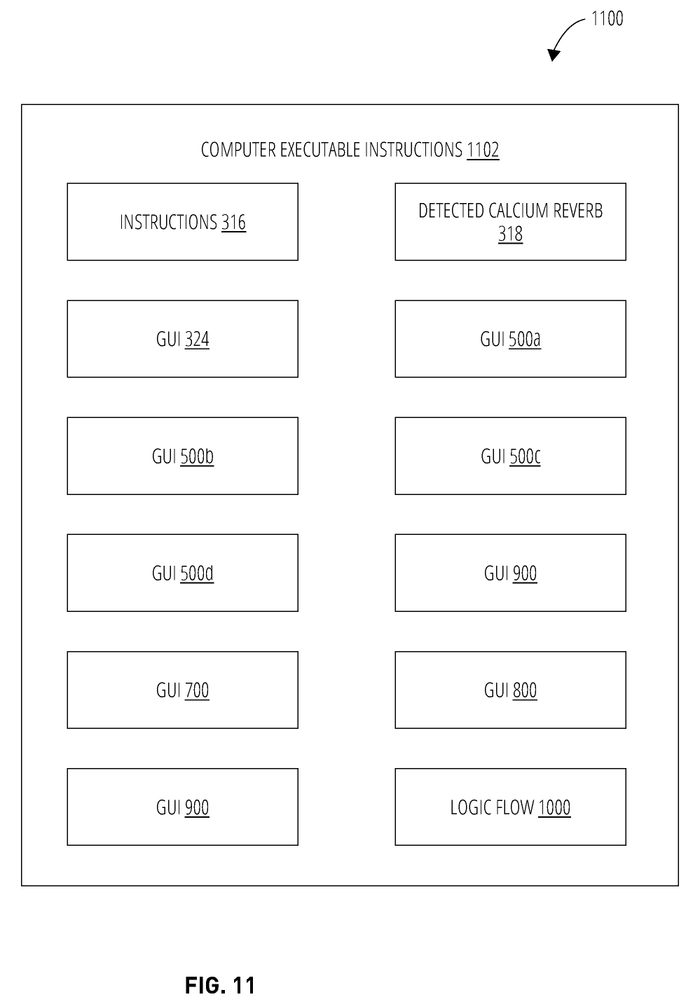

FIG. 11 illustrates computer-readable storage medium 1100. Computer-readable storage medium 1100 may comprise any non-transitory computer-readable storage medium or machine-readable storage medium, such as an optical, magnetic or semiconductor storage medium. In various embodiments, computer-readable storage medium 1100 may comprise an article of manufacture. In some embodiments, computer-readable storage medium 1100 may store computer executable instructions 1102 with which circuitry (e.g., processor 124, processor 306, IVUS imaging system acquisition circuitry 314, and the like) can execute. For example, computer executable instructions 1102 can include instructions to implement operations described with respect to instructions 316, logic flow 1000, detected calcium reverb 318, GUI 324, GUI 500a, GUI 500b, GUI 500c, GUI 500d, GUI 900, GUI 700, GUI 800, GUI 900, and/or logic flow 1000. Examples of computer-readable storage medium 1100 or machine-readable storage medium may include any tangible media capable of storing electronic data, including volatile memory or non-volatile memory, removable or non-removable memory, erasable or non-erasable memory, writeable or re-writeable memory, and so forth. Examples of computer executable instructions 1102 may include any suitable type of code, such as source code, compiled code, interpreted code, executable code, static code, dynamic code, object-oriented code, visual code, and the like.

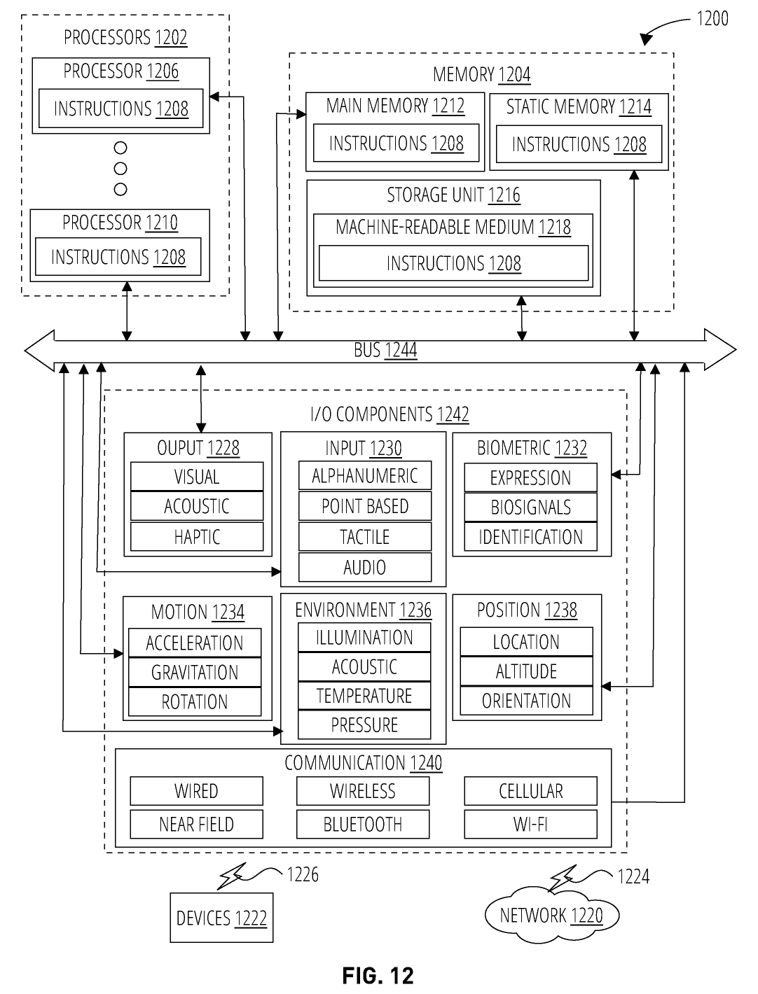

FIG. 12 illustrates a diagrammatic representation of a machine 1200 in the form of a computer system within which a set of instructions may be executed for causing the machine to perform any one or more of the methodologies discussed herein. More specifically, FIG. 12 shows a diagrammatic representation of the machine 1200 in the example form of a computer system, within which instructions 1208 (e.g., software, a program, an application, an applet, an app, or other executable code) for causing the machine 1200 to perform any one or more of the methodologies discussed herein may be executed. For example, the instructions 1208 may cause the machine 1200 to execute logic flow 1000 of FIG. 10, instructions 316 of FIG. 3. More generally, the instructions 1208 may cause the machine 1200 to generate GUIs with functionality and behavior as described herein during a pre-PCI, peri-PCI, or post-PCI using IVUS. It is noted that the present disclosure provides specific and discrete implementations of GUI representations and behavior that is a significant improvement over the prior art. In particular, the present disclosure provides an improvement to computing technology in that GUIs provide greater visibility and navigation of IVUS images including notation of locations of calcium reverb.

The instructions 1208 transform the general, non-programmed machine 1200 into a particular machine 1200 programmed to carry out the described and illustrated functions in a specific manner. In alternative embodiments, the machine 1200 operates as a standalone device or may be coupled (e.g., networked) to other machines. In a networked deployment, the machine 1200 may operate in the capacity of a server machine or a client machine in a server-client network environment, or as a peer machine in a peer-to-peer (or distributed) network environment. The machine 1200 may comprise, but not be limited to, a server computer, a client computer, a personal computer (PC), a tablet computer, a laptop computer, a netbook, a set-top box (STB), a PDA, an entertainment media system, a cellular telephone, a smart phone, a mobile device, a wearable device (e.g., a smart watch), a smart home device (e.g., a smart appliance), other smart devices, a web appliance, a network router, a network switch, a network bridge, or any machine capable of executing the instructions 1208, sequentially or otherwise, that specify actions to be taken by the machine 1200. Further, while only a single machine 1200 is illustrated, the term “machine” shall also be taken to include a collection of machines 1200 that individually or jointly execute the instructions 1208 to perform any one or more of the methodologies discussed herein.

The machine 1200 may include processors 1202, memory 1204, and I/O components 1242, which may be configured to communicate with each other such as via a bus 1244. In an example embodiment, the processors 1202 (e.g., a Central Processing Unit (CPU), a Reduced Instruction Set Computing (RISC) processor, a Complex Instruction Set Computing (CISC) processor, a Graphics Processing Unit (GPU), a Digital Signal Processor (DSP), an ASIC, a Radio-Frequency Integrated Circuit (RFIC), another processor, or any suitable combination thereof) may include, for example, a processor 1206 and a processor 1210 that may execute the instructions 1208. The term “processor” is intended to include multi-core processors that may comprise two or more independent processors (sometimes referred to as “cores”) that may execute instructions contemporaneously. Although FIG. 12 shows multiple processors 1202, the machine 1200 may include a single processor with a single core, a single processor with multiple cores (e.g., a multi-core processor), multiple processors with a single core, multiple processors with multiples cores, or any combination thereof.

The memory 1204 may include a main memory 1212, a static memory 1214, and a storage unit 1216, both accessible to the processors 1202 such as via the bus 1244. The main memory 1204, the static memory 1214, and storage unit 1216 store the instructions 1208 embodying any one or more of the methodologies or functions described herein. The instructions 1208 may also reside, completely or partially, within the main memory 1212, within the static memory 1214, within machine-readable medium 1218 within the storage unit 1216, within at least one of the processors 1202 (e.g., within the processor’s cache memory), or any suitable combination thereof, during execution thereof by the machine 1200.

The I/O components 1242 may include a wide variety of components to receive input, provide output, produce output, transmit information, exchange information, capture measurements, and so on. The specific I/O components 1242 that are included in a particular machine will depend on the type of machine. For example, portable machines such as mobile phones will likely include a touch input device or other such input mechanisms, while a headless server machine will likely not include such a touch input device. It will be appreciated that the I/O components 1242 may include many other components that are not shown in FIG. 12. The I/O components 1242 are grouped according to functionality merely for simplifying the following discussion and the grouping is in no way limiting. In various example embodiments, the I/O components 1242 may include output components 1228 and input components 1230. The output components 1228 may include visual components (e.g., a display such as a plasma display panel (PDP), a light emitting diode (LED) display, a liquid crystal display (LCD), a projector, or a cathode ray tube (CRT)), acoustic components (e.g., speakers), haptic components (e.g., a vibratory motor, resistance mechanisms), other signal generators, and so forth. The input components 1230 may include alphanumeric input components (e.g., a keyboard, a touch screen configured to receive alphanumeric input, a photo-optical keyboard, or other alphanumeric input components), point-based input components (e.g., a mouse, a touchpad, a trackball, a joystick, a motion sensor, or another pointing instrument), tactile input components (e.g., a physical button, a touch screen that provides location and/or force of touches or touch gestures, or other tactile input components), audio input components (e.g., a microphone), and the like.

In further example embodiments, the I/O components 1242 may include biometric components 1232, motion components 1234, environmental components 1236, or position components 1238, among a wide array of other components. For example, the biometric components 1232 may include components to detect expressions (e.g., hand expressions, facial expressions, vocal expressions, body gestures, or eye tracking), measure bio signals (e.g., blood pressure, heart rate, body temperature, perspiration, or brain waves), identify a person (e.g., voice identification, retinal identification, facial identification, fingerprint identification, or electroencephalogram-based identification), and the like. The motion components 1234 may include acceleration sensor components (e.g., accelerometer), gravitation sensor components, rotation sensor components (e.g., gyroscope), and so forth. The environmental components 1236 may include, for example, illumination sensor components (e.g., photometer), temperature sensor components (e.g., one or more thermometers that detect ambient temperature), humidity sensor components, pressure sensor components (e.g., barometer), acoustic sensor components (e.g., one or more microphones that detect background noise), proximity sensor components (e.g., infrared sensors that detect nearby objects), gas sensors (e.g., gas detection sensors to detection concentrations of hazardous gases for safety or to measure pollutants in the atmosphere), or other components that may provide indications, measurements, or signals corresponding to a surrounding physical environment. The position components 1238 may include location sensor components (e.g., a GPS receiver component), altitude sensor components (e.g., altimeters or barometers that detect air pressure from which altitude may be derived), orientation sensor components (e.g., magnetometers), and the like.

Communication may be implemented using a wide variety of technologies. The I/O components 1242 may include communication components 1240 operable to couple the machine 1200 to a network 1220 or devices 1222 via a coupling 1224 and a coupling 1226, respectively. For example, the communication components 1240 may include a network interface component or another suitable device to interface with the network 1220. In further examples, the communication components 1240 may include wired communication components, wireless communication components, cellular communication components, Near Field Communication (NFC) components, Bluetooth^{®} components (e.g., Bluetooth^{®} Low Energy), Wi-Fi^{®} components, and other communication components to provide communication via other modalities. The devices 1222 may be another machine or any of a wide variety of peripheral devices (e.g., a peripheral device coupled via a USB).

Moreover, the communication components 1240 may detect identifiers or include components operable to detect identifiers. For example, the communication components 1240 may include Radio Frequency Identification (RFID) tag reader components, NFC smart tag detection components, optical reader components (e.g., an optical sensor to detect one-dimensional bar codes such as Universal Product Code (UPC) bar code, multi-dimensional bar codes such as Quick Response (QR) code, Aztec code, Data Matrix, Dataglyph, MaxiCode, PDF417, Ultra Code, UCC RSS-2D bar code, and other optical codes), or acoustic detection components (e.g., microphones to identify tagged audio signals). In addition, a variety of information may be derived via the communication components 1240, such as location via Internet Protocol (IP) geolocation, location via Wi-Fi® signal triangulation, location via detecting an NFC beacon signal that may indicate a particular location, and so forth.

The various memories (i.e., memory 1204, main memory 1212, static memory 1214, and/or memory of the processors 1202) and/or storage unit 1216 may store one or more sets of instructions and data structures (e.g., software) embodying or utilized by any one or more of the methodologies or functions described herein. These instructions (e.g., the instructions 1208), when executed by processors 1202, cause various operations to implement the disclosed embodiments.

As used herein, the terms “machine-storage medium,” “device-storage medium,” “computer-storage medium” mean the same thing and may be used interchangeably in this disclosure. The terms refer to a single or multiple storage devices and/or media (e.g., a centralized or distributed database, and/or associated caches and servers) that store executable instructions and/or data. The terms shall accordingly be taken to include, but not be limited to, solid-state memories, and optical and magnetic media, including memory internal or external to processors. Specific examples of machine-storage media, computer-storage media and/or device-storage media include non-volatile memory, including by way of example semiconductor memory devices, e.g., erasable programmable read-only memory (EPROM), electrically erasable programmable read-only memory (EEPROM), FPGA, and flash memory devices; magnetic disks such as internal hard disks and removable disks; magneto-optical disks; and CD-ROM and DVD-ROM disks. The terms “machine-storage media,” “computer-storage media,” and “device-storage media” specifically exclude carrier waves, modulated data signals, and other such media, at least some of which are covered under the term “signal medium” discussed below.

In various example embodiments, one or more portions of the network 1220 may be an ad hoc network, an intranet, an extranet, a VPN, a LAN, a WLAN, a WAN, a WWAN, a MAN, the Internet, a portion of the Internet, a portion of the PSTN, a plain old telephone service (POTS) network, a cellular telephone network, a wireless network, a Wi-Fi® network, another type of network, or a combination of two or more such networks. For example, the network 1220 or a portion of the network 1220 may include a wireless or cellular network, and the coupling 1224 may be a Code Division Multiple Access (CDMA) connection, a Global System for Mobile communications (GSM) connection, or another type of cellular or wireless coupling. In this example, the coupling 1224 may implement any of a variety of types of data transfer technology, such as Single Carrier Radio Transmission Technology (1xRTT), Evolution-Data Optimized (EVDO) technology, General Packet Radio Service (GPRS) technology, Enhanced Data rates for GSM Evolution (EDGE) technology, third Generation Partnership Project (3GPP) including 3G, fourth generation wireless (4G) networks, Universal Mobile Telecommunications System (UMTS), High Speed Packet Access (HSPA), Worldwide Interoperability for Microwave Access (WiMAX), Long Term Evolution (LTE) standard, others defined by various standard-setting organizations, other long range protocols, or other data transfer technology.

The instructions 1208 may be transmitted or received over the network 1220 using a transmission medium via a network interface device (e.g., a network interface component included in the communication components 1240) and utilizing any one of several well-known transfer protocols (e.g., hypertext transfer protocol (HTTP)). Similarly, the instructions 1208 may be transmitted or received using a transmission medium via the coupling 1226 (e.g., a peer-to-peer coupling) to the devices 1222. The terms “transmission medium” and “signal medium” mean the same thing and may be used interchangeably in this disclosure. The terms “transmission medium” and “signal medium” shall be taken to include any intangible medium that is capable of storing, encoding, or carrying the instructions 1208 for execution by the machine 1200, and includes digital or analog communications signals or other intangible media to facilitate communication of such software. Hence, the terms “transmission medium” and “signal medium” shall be taken to include any form of modulated data signal, carrier wave, and so forth. The term “modulated data signal” means a signal that has one or more of its characteristics set or changed in such a matter as to encode information in the signal.

Terms used herein should be accorded their ordinary meaning in the relevant arts, or the meaning indicated by their use in context, but if an express definition is provided, that meaning controls.

Herein, references to "one embodiment" or "an embodiment" do not necessarily refer to the same embodiment, although they may. Unless the context clearly requires otherwise, throughout the description and the claims, the words "comprise," "comprising," and the like are to be construed in an inclusive sense as opposed to an exclusive or exhaustive sense; that is to say, in the sense of "including, but not limited to." Words using the singular or plural number also include the plural or singular number respectively, unless expressly limited to one or multiple ones. Additionally, the words "herein," "above," "below" and words of similar import, when used in this application, refer to this application as a whole and not to any portions of this application. When the claims use the word "or" in reference to a list of two or more items, that word covers all the following interpretations of the word: any of the items in the list, all the items in the list and any combination of the items in the list, unless expressly limited to one or the other. Any terms not expressly defined herein have their conventional meaning as commonly understood by those having skill in the relevant art(s).

By using genuine models of anatomy more accurate surgical plans may be developed than through statistical modeling.

Terms used herein should be accorded their ordinary meaning in the relevant arts, or the meaning indicated by their use in context, but if an express definition is provided, that meaning controls.

Herein, references to "one embodiment" or "an embodiment" do not necessarily refer to the same embodiment, although they may. Unless the context clearly requires otherwise, throughout the description and the claims, the words "comprise," "comprising," and the like are to be construed in an inclusive sense as opposed to an exclusive or exhaustive sense; that is to say, in the sense of "including, but not limited to." Words using the singular or plural number also include the plural or singular number respectively, unless expressly limited to one or multiple ones. Additionally, the words "herein," "above," "below" and words of similar import, when used in this application, refer to this application as a whole and not to any portions of this application. When the claims use the word "or" in reference to a list of two or more items, that word covers all the following interpretations of the word: any of the items in the list, all the items in the list and any combination of the items in the list, unless expressly limited to one or the other. Any terms not expressly defined herein have their conventional meaning as commonly understood by those having skill in the relevant art(s).

By using genuine models of anatomy more accurate surgical plans may be developed than through statistical modeling.

Terms used herein should be accorded their ordinary meaning in the relevant arts, or the meaning indicated by their use in context, but if an express definition is provided, that meaning controls.

Herein, references to "one embodiment" or "an embodiment" do not necessarily refer to the same embodiment, although they may. Unless the context clearly requires otherwise, throughout the description and the claims, the words "comprise," "comprising," and the like are to be construed in an inclusive sense as opposed to an exclusive or exhaustive sense; that is to say, in the sense of "including, but not limited to." Words using the singular or plural number also include the plural or singular number respectively, unless expressly limited to one or multiple ones. Additionally, the words "herein," "above," "below" and words of similar import, when used in this application, refer to this application as a whole and not to any portions of this application. When the claims use the word "or" in reference to a list of two or more items, that word covers all the following interpretations of the word: any of the items in the list, all the items in the list and any combination of the items in the list, unless expressly limited to one or the other. Any terms not expressly defined herein have their conventional meaning as commonly understood by those having skill in the relevant art(s).

What is claimed is:

1. A vascular imaging system, comprising:
a display;
a processor coupled to the display; and
a memory coupled to the processor, the memory comprising instructions executable by the processor, which instructions when executed cause the processor to:
receive a series of intravascular ultrasound (IVUS) images of a vessel of a patient, the series of IVUS images comprising a plurality of frames;
detect calcium reverb from the series of IVUS images;
generate a graphical component comprising an indication of the calcium reverb relative to the series of IVUS images;
generate a GUI comprising a cross-sectional view of a frame of the series of IVUS images and the graphical component showing the calcium reverb relative to the frame; and
render the GUI for display on the display.

2. The vascular imaging system of claim 1, wherein the instructions when execute by the processor further cause the processor to place the graphical component around a circumference of the vessel depicted in the frame corresponding to the location of the calcium reverb.

3. The vascular imaging system of claim 1, wherein the instructions when execute by the processor further cause the processor to:
generate a longitudinal view of the series of IVUS images; and
generate the graphical component comprising the cross-sectional view, the graphical component, and the longitudinal view.

4. The vascular imaging system of claim 3, wherein the longitudinal view comprises a slider and wherein the position of the slider along a longitudinal axis of the of the vessel corresponds to the frame.

5. The vascular imaging system of claim 3, wherein the instructions when execute by the processor further cause the processor to:
generate a vessel profile view from the series of IVUS images; and
generate the graphical component comprising the cross-sectional view, the graphical component, the longitudinal view, and the vessel profile view.

6. The vascular imaging system of claim 5, wherein the vessel profile view comprises an indication of the lumen border and the vessel border along the length of the vessel represented by the series of IVUS images.

7. The vascular imaging system of claim 6, wherein the vessel profile view further comprises an indication of one or more key frames.

8. The vascular imaging system of claim 6, wherein the instructions when execute by the processor further cause the processor to:
detect calcium from the series of IVUS images,
wherein the vessel profile view further comprises an indication of the length of the detected calcium along a longitudinal axis of the vessel.

9. The vascular imaging system of claim 8, wherein the instructions when execute by the processor further cause the processor to:
detect an arc length of calcium from the series of IVUS images,
wherein the vessel profile view further comprises an indication of areas where the arc length of the calcium is greater than 360°.

10. The vascular imaging system of claim 9, wherein the longitudinal view and/or the vessel profile comprises the graphical component.

11. At least one machine readable storage device, comprising a plurality of instructions that in response to being executed by a processor of a vascular imaging system cause the processor to:
receive a series of intravascular ultrasound (IVUS) images of a vessel of a patient, the series of IVUS images comprising a plurality of frames;
detect calcium reverb from the series of IVUS images;
generate a graphical component comprising an indication of the calcium reverb relative to the series of IVUS images;
generate a GUI comprising a cross-sectional view of a frame of the series of IVUS images and the graphical component showing the calcium reverb relative to the frame; and
render the GUI for display on the display.

12. The at least one machine readable storage device of claim 11, wherein the graphical component comprises a plurality of graphical components each indicating a respective calcium reverberation.

13. The at least one machine readable storage device of claim 11, wherein generating the GUI comprises placing the plurality of graphical component around a circumference of the vessel depicted in the frame corresponding to locations of the respective calcium reverberations.

14. The at least one machine readable storage device of claim 11, wherein the indication of the calcium reverb is a circular icon with a central geometric feature and lines emanating from the central geometric feature or wherein the calcium reverb icon is a featured line.

15. A method, comprising:
receiving a series of intravascular ultrasound (IVUS) images of a vessel of a patient, the series of IVUS images comprising a plurality of frames;
detecting calcium reverb from the series of IVUS images;
generating a graphical component comprising an indication of the calcium reverb relative to the series of IVUS images;
generating a GUI comprising a cross-sectional view of a frame of the series of IVUS images and the graphical component showing the calcium reverb relative to the frame; and
rendering the GUI for display on a display.

16. The method of claim 15, wherein generating the GUI comprises placing the graphical component around a circumference of the vessel depicted in the frame corresponding to the location of the calcium reverb.

17. The method of claim 15, further comprising:
generating a longitudinal view of the series of IVUS images; and
generating the graphical component comprising the cross-sectional view, the graphical component, and the longitudinal view.

18. The method of claim 17, wherein the longitudinal view comprises a slider and wherein the position of the slider along a longitudinal axis of the of the vessel corresponds to the frame.

19. The method of claim 17, further comprising:
generating a vessel profile view from the series of IVUS images; and
generating the graphical component comprising the cross-sectional view, the graphical component, the longitudinal view, and the vessel profile view.

20. The method of claim 19, wherein the vessel profile view comprises an indication of the lumen border and the vessel border along the length of the vessel represented by the series of IVUS images.
